(19) United States
(12) Patent Application Publication
(10) Pub. No.: US 2008/0299542 A1
(43) Pub. Date: Dec. 4, 2008

(54) IDENTIFICATION OF OXIDATIVELY MODIFIED PEPTIDE SEQUENCES IN THE PROTEOME (LOSCALZO)

(76) Inventor: Joseph Loscalzo, Dover, MA (US)

Correspondence Address: WOLF GREENFIELD & SACKS, P.C., 600 ATLANTIC AVENUE, BOSTON, MA, 02210-2206, US

(21) Appl. No.: 12/120,501

(22) Filed: May 14, 2008

Related U.S. Application Data
(60) Provisional application No. 60/938,069, filed on May 15, 2007.

(51) Int. Cl. C12Q 1/00; G01N 33/00

(52) U.S. Cl. 435/4; 436/86

(57) ABSTRACT

The invention relates, in part, to identifying protein target(s) that undergo oxidative modification and the specific peptide sequences bearing oxidation-induced modifications.

### RELATED APPLICATION

This application claims the benefit under 35 U.S.C. §119(e) of U. S. provisional application Ser. No. 60/938,069, filed May 15, 2007, the entire contents of which are incorporated by reference herein.

### GOVERNMENT SUPPORT

This invention was made in part with government support under grant numbers HL61795, HL58976, HL55993, and HV28178 from the National Institute of Health. The United States government may have certain rights in this invention.

### FIELD OF THE INVENTION

The invention relates to identifying protein targets that undergo oxidative modification.

### BACKGROUND OF THE INVENTION

Proteins are responsible for biological form and function. It is estimated that there are ˜6 to 7 times as many distinct proteins as genes in humans, in part owing to splicing and exchange of various structural cassettes among genes during transcription. Proteins are not only more abundant than the genes that encode them, but they are also much more structurally complex with primary, secondary, tertiary, and quaternary structural elements. Additionally, proteins have greatly varied biochemical functions that critically depend on structure. Furthermore, mature proteins are also subject to a host of post-translational modifications, including proteolysis, sulfhydryl oxidation and disulfide bond formation, phosphorylation, glycosylation, S-nitrosation, fatty acylation, and oxidation. These biochemical modifications often yield products with functions different from those of the unmodified parent protein, and many of these modifications, such as oxidation, reflect the consequences of environmental modulation of genetic determinants. The post-translational changes in protein structure add more complexity to the primary protein sequence coded by the genome and, together with the primary sequence of all proteins, comprise the proteome.

Proteomics, the study of the proteome, has been defined as the sequence, modification, and function of all proteins in a biological system. Proteomics is in its infancy, yet it will prove to be essential to understanding human biology and disease. Proteomics, has the potential to revolutionize how we diagnose disease, assess risk, determine prognosis, and target therapeutic strategies among individuals with various diseases or disorders. It is hoped that analysis of a blood sample will provide unique prognostic information about a subject's risk for a disease or about the prognosis of a disease or disorder. In addition, understanding the proteome will improve our understanding of protein function and will allow tailoring therapeutic strategies to correct altered function or enhance the basal function of a specific protein or a set of proteins.

### SUMMARY OF THE INVENTION

The majority of protein disulfides in cells are considered an important inert structural, rather than a dynamic regulatory, determinant of protein function. Applicant has discovered that some disulfides in proteins are also regulated by cell redox status with functional consequences. The invention is directed, in part, to identifying protein targets that undergo oxidative modification and the specific peptide sequences bearing those oxidatively modified side chains. Although not intending to be bound by any particular mechanism or theory, the working hypothesis is that G6PD and its enzymatic product, NADPH, (a) regulate the thiol redox state of the cell, (b) are essential for normal oxidant signaling and cell function, and (c) adaptively respond to increased reactive oxygen species (ROS) generation to maintain a state of compensated oxidant stress. This hypothesis incorporates the concepts that increasing ROS flux produces different patterns of oxidative and nitrosative modifications of critical protein thiols; that these patterns can serve as molecular signatures that distinguish among states of oxidant signaling, compensated oxidant stress, and uncompensated oxidant stress in cells, and that G6PD and NADPH regulate, in part, the transitions among these states. Potential consensus sequences may also promote thiol oxidation (e.g., by hydrogen peroxide) owing to their propensity to maintain the thiol in the thiolate (S⁻) oxidation state and its ability to react with hydrogen peroxide via an SN₂ mechanism. The last four amino acids (RKCF) of the carboxy-terminal of actin comprise an example of such a sequence.

According to one aspect of the invention, a method of identifying an oxidation-modified protein wherein an amino acid has undergone an oxidation-induced modification is provided. The method involves determining an oxidation state of the protein, exposing the protein to a condition that results in oxidation-induced modification of an amino acid in the protein, and determining the oxidation state of the protein after exposure to the condition that results in oxidation-induced modification of an amino acid in the protein. The oxidation states of the protein before and after exposure to the condition that results in oxidation-induced modification of an amino acid in the protein are compared. If the oxidation state of the protein after exposure to the condition that results in oxidation-induced modification of an amino acid in the protein is greater than the oxidation state of the protein before exposure to the condition that results in oxidation-induced modification of an amino acid in the protein, the protein is identified as an oxidation-modified protein.

The amino acid in the protein may be cysteine, methionine, arginine, or tryptophane. The oxidatively-modified peptide in the oxidation-modified protein may be identified by mass spectrometry. In some embodiments, the oxidation state comprises the oxidation state of a thiol pool. The thiol may be a protein vicinal dithiol (Pr(SH)₂) pool, a protein glutathiolated (PrSSG) pool, or an interprotein disulfide pool (PrSSPr′) in the sample. In some embodiments, the oxidation state comprises a level of S-nitrosylation or a level of cysteine oxidation.

In some embodiments, the oxidation-induced modification comprises a modification generated in a disease or a disorder. In some embodiments, the condition that results in an oxidation-induced modification comprises hydrogen peroxide, superoxide, peroxynitrite or perchlorate.

According to another aspect of the invention, a method of diagnosing a disorder or predicting the risk of developing a disorder characterized or caused by oxidant stress in a subject is provided. The method involves determining an oxidation state in a protein and comparing the oxidation state in the protein to a control. An increase in the oxidation state compared to the control indicates that the subject has a disorder or is at risk of developing a disorder characterized or caused by oxidant stress. The disorder may be an inflammatory disorder, an auto-immune disorder, a cardiovascular disorder, or insulin-independent diabetes mellitus (type II diabetes).

Examples of inflammatory disorders include but are not limited to allergic rhinitis, ankylosing spondilitis, arthritis, asthma, Behcet syndrome, bursitis, chronic obstructive pulmonary disease (COPD), Churg-Strauss syndrome, dermatitis, gout, Henoch-Schonlein purpura, inflammatory bowel disease (Crohn's disease or ulcerative colitis), inflammatory neuropathy, Kawasaki disease, myositis, neuritis, pericardits, polyarteritis nodosa, polymyalgia rheumatica, prostatitis, psoriasis, radiation injury, sarcoidosis, shock, sytemic inflammatory response syndrome (SIRS), Takayasu's arteritis, temporal arteritis, thromboangiitis obliterans (Buerger's disease), vasculitis, and Wegener's granulomatosus.

Examples of autoimmune disorders include but are not limited to Addison's disease, chronic thyroiditis, dermatomyositis, Grave's disease, Hashimoto's thyroiditis, hypersensitivity pneumonitis, insulin-dependent diabetes mellitus (type I diabetes), multiple sclerosis, myasthenia gravis, organ transplantation, pernicious anemia, Reiter's syndrome, rheumatoid arthritis, Sjogren's syndrome, systemic lupus erythematosis (SLE), thyroiditis, and urticaria.

Examples of cardiovascular disorders include but are not limited to coronary artery disease, ischemic cardiomyopathy, myocardial ischemia, ischemic or post-myocardial ischemia revascularization, diabetic retinopathy, diabetic nephropathy, renal fibrosis, hypertension, atherosclerosis, arteriosclerosis, atherosclerotic plaque, atherosclerotic plaque rupture, cerebrovascular accident (stroke), transient ischemic attack (TIA), peripheral artery disease, arterial occlusive disease, vascular aneurysm, ischemia, ischemic ulcer, heart valve stenosis, heart valve regurgitation and intermittent claudication.

In some embodiments, the protein is in a sample from the subject. The sample may be blood, serum, plasma, urine, sputum, saliva, stool, cerebrospinal fluid, peritoneal fluid, cell, tissue, or a secretion.

According to yet another aspect of the invention, a method of screening for an agent that modulates an oxidation state of a protein is provided. The method comprises determining an oxidation state of the protein, exposing the protein to an agent or a condition that results in an oxidation-induced modification of an amino acid side in the protein, and determining the oxidation state of the protein after exposing the protein to the agent.

The oxidation states of the protein before and after exposure to the condition that results in oxidation-induced modification of an amino acid in the protein are compared. If the oxidation state of the protein after exposure to the condition that results in oxidation-induced modification of an amino acid in the protein is greater than the oxidation state of the protein before exposure to the condition that results in oxidation-induced modification of an amino acid in the protein, the agent or condition is an oxidant or a pro-oxidant. If the oxidation state of the protein after exposure to the condition that results in oxidation-induced modification of an amino acid in the protein is less than the oxidation state of the protein before exposure to the condition that results in oxidation-induced modification of an amino acid in the protein, the agent or condition is an anti-oxidant.

These and other aspects of the invention, as well as various advantages and utilities, will be more apparent with reference to the detailed description of the invention. Each aspect of the invention can encompass various embodiments as will be understood.

### BRIEF DESCRIPTION OF THE DRAWINGS

FIG. 1. a, is a fluorescent image of protein disulfides in Chang liver cells after treatment with CCCP for 4 hr. b, is a histogram showing the effect of different mitochondrial inhibitors on disulfide-containing proteins (filled bars) and superoxide generation (open bars) measured by DHE fluorescence in BAECs: a) control, b) rotenone, c) myxothiazol, d) TTFA, e) ATM, and f) CCCP. Protein disulfide staining was semi-quantified with fluorescence microscopy and ImageJ software. c, is a histogram showing the effect of inhibitors of catalase (3-amino-1,2,4-triazole, 3-AT) and GPx (beta-mercaptosuccinic acid, MS) and of a GPx-mimetic, ebselen, on protein disulfide content. d, is an immunofluorescence image showing the effect of antioxidant enzyme overexpression on protein disulfide formation. Cells were infected with 25 MOI adenovirus containing the following genes: MnSOD, manganese superoxide dismutase; CAT, wild type catalase; MitoCAT, catalase targeted to mitochondria. e, is a fluorescent image showing that protein disulfide formation decreases in Rho₀ cells devoid of functional mitochondria (27). f, is a blot of biotin-labeled disulfide-containing proteins. Protein disulfides were labeled with either MTSEA-biotin or BIAM, and then detected with streptavidin-conjugated HRP. Lane 1, control; Lane 2, CCCP; Lane 3, ATM. Cells were treated with mitochondrial inhibitors for 8 hr. g, is an immunoblot of disulfide-containing and total actin and CD98. h, is a blot showing heterodimer formation of CD98 in mitochondrial inhibitor-treated cells. i, Immunoblot of multimeric vWF in HPAECs after mitochondrial inhibitor treatment. Lane 1, control; Lane 2, CCCP; Lane 3, ATM; Lane 4, TTFA; Lane 5, DHEA; Lane 6, BSO. j, is an immunoblot of endoglin on a nonreducing gel. Antibody used was a mouse monoclonal anti-P4A4 antibody or a rabbit polyclonal anti-H300 antibody. k, is an immunofluorescence image of endoglin, PECAM, and vWF in HPAECs after treatment with mitochondrial inhibitors. The Golgi was stained with Alexa 350-labeled WGA in the endoglin experiment, while nuclei were stained with DAPI in the PECAM and vWF experiments. l, is a blot showing GRP78 and GRP94 protein induction by mitochondrial inhibitors, tunicamycin, or thapsagargin.

FIG. 2 shows the response of cellular protein disulfide to oxidative stress. a, is a fluorescent image of the effect of hydrogen peroxide on protein disulfides in BAECs. Cells were treated with a range of concentrations of hydrogen peroxide (0-8 mM) for 30 min. b, is an immunofluorescence image showing the effect of glucose-6-phosphate dehydrogenase inhibition (to inhibit NADPH generation for glutathione reductase, and, thereby, GPx activity, increasing cellular oxidant stress) on protein disulfides in BAECs. Cells were treated with 0.1 mM DHEA for 24 hr.

FIG. 3 is a histogram of protein disulfide fluorescence. Cells were treated with different ROS-generating enzyme inhibitors for 8 hrs before staining for protein disulfide. a, control; b, L-NAME for nitric oxide synthases; c, nialamide for monoamine oxidase; d, indomethacin for cyclooxygenase; e, allopurinol for xanthine oxidase; f, DIDS for VDAC ion channel; g, 1-aminobenzotriazole for cytochrome p450; and h, apocynin for NADPH oxidase. N=3 experiments each performed in triplicate.

FIG. 4 is a histogram of protein disulfide fluorescence. showing the effect of antioxidant enzyme on disulfide-containing proteins. Cells were treated with 25 MOI adenovirus containing the MnSOD, CAT, or MitoCAT gene and cultured for 48 hrs before measurements. a, MitoCAT, but not MnSOD or CAT, markedly decreased the protein disulfide signal. b, MitoCAT-overexpressing cells were treated with CCCP for 8 hrs before protein disulfides staining and compared with control MitoCAT-overexpressing cells not treated with CCCP.

FIG. 5 shows the effect of mitochondrial inhibitors on various growth factor receptors. Cells were treated with mitochondrial inhibitors for 8 hr before assays. a, Immunofluorescence image of uptake of Alexa 488-labeled EGF by Chang liver cells or AcLDL by HPAECs. Nuclei were counterstained with DAPI. b, Graph of time-dependent decrease of AcLDL uptake (filled circles) and mitochondrial membrane potential (open circles) in HPAECs after treatment with CCCP. Mitochondrial potential was measured by fluorescence of JC-1 dye expressed as the ratio of emission at 590 nm and at 536 nm. To measure cell surface receptor autophosphorylation, cells were incubated with 100 ng/ml EGF (c), 25 ng/ml IGF-1 (d), or 25 ng/ml bFGF (e) for 5 min before lysis and analysis by immunoblot.

FIG. 6 shows the effect of mitochondrial inhibitors on expression of different receptors and redox enzymes. Cells were treated with mitochondrial inhibitors for 8 hr. a, Chang liver cells; b, HPAECs.

FIG. 7 is a histogram showing the effect of mitochondrial inhibitors on transferrin uptake by HPAECs. Cells were treated with mitochondrial inhibitors for 8 hr. After labeling with Alexa 488-labeled transferrin, cells were trypsin-treated and analyzed by flow cytometry. N=3 experiments each performed in triplicate.

FIG. 8 shows the alteration of cell-surface receptor function by direct disulfide reduction. Near confluent cells were treated with different concentrations of DTT in growth medium for 30 min before receptor assays. a, Quantification of AcLDL and transferrin uptake by HPAECs and of EGF uptake by Chang liver cells after 0.5 mM DTT treatment by flow cytometry. b, Ligand-induced phosphorylation of EGFR (b), IGF-1β (c), and FGFR1 (d) in Chang liver cells after DTT treatment.

FIG. 9 shows cell density-dependence of the disulfide proteome. Chang liver cells or HPAECs were seeded at 1,000 cell/cm² or 10,000 cell/cm², respectively, and cultured for 2 days. a, Comparison of disulfide staining and immunofluorescence of endoglin and vWF in sparse and confluent HPAECs. Cells were counterstained with WGA in endoglin experiments or DAPI in vWF experiments. b, Sparse Chang liver cells are in a more reductive state than confluent cells, as indicated by GSH staining, mitochondrial membrane potential measured by JC-1 fluorescence, and superoxide generation measured by MitoSox staining. Nuclei were counterstained with Hoechst 33342. c, EGF binding to Chang liver cells and AcLDL uptake by HPAEC. Cell nuclei were counterstained with DAPI. d, EGF phosphorylation of EGFR after EGF incubation was detected as in FIG. 5. e, IGF-1β phosphorylation in Chang liver cells after incubation with IGF-1.

### DETAILED DESCRIPTION OF THE INVENTION

Oxidants such as hydrogen peroxide (H₂O₂) are implicated in mediating a wide array of human diseases. Oxidants contribute to disease processes by causing damage to biomolecules and by altering cellular metabolism. Proteins are among the targets for oxidative modification and/or damage. In order to understand how oxidative stress can cause disease, it is important to discover which proteins become affected by oxidative stress, how they are modified, and the functional consequences of the modifications.

This invention is directed, in part, to identifying protein target(s) (oxidation-modified protein(s)) that undergo oxidative modification and the specific peptide sequences bearing those oxidation-induced modifications. The invention involves determining the oxidation state of the protein before and after exposure to a condition that results in oxidation-induced modification of an amino acid in the protein and comparing these oxidation states. All peptide side chains may be examined for oxidation-induced modification. In some embodiments, side chains having thiol groups will be examined for oxidative modification. Oxidation-induced modifications may be reversible or irreversible. The modified sequences are independent of the underlying coded sequence as DNA sequences cannot predict posttranslational oxidative modification.

An “oxidation-modified protein” is a protein that has been exposed or subjected to oxidation. Oxidation describes the loss of electrons by a molecule (e.g., protein) or an atom in the molecule. Oxidation causes an increase in the charge (oxidation number) of a molecule (e.g., protein) or an atom.

An “oxidation-induced modification” of a protein refers to a change that causes an increase in the charge (oxidation number) of a protein or of an amino acid in the protein. Thus, a change in the oxidation state of a amino acid is reflected as a change in the charge of the protein or of the amino acid. A loss of one or more electrons by a amino acid (typically induced by an oxidant) will result in an increase in the oxidation state of the protein or amino acid. A gain of one or more electrons (typically induced by a reductant) will result in an decrease in the oxidation state of the protein or amino acid.

An oxidant is an agent that oxidizes another molecule (e.g., one or more amino acids in a protein). Typically, an oxidant is a molecule that accepts one or more electrons. Examples of oxidants include but are not limited to hydrogen peroxide, superoxide, peroxynitrite, and perchlorate. Examples of oxidants also include nitric oxide (NO), NO-related species, oxygen related species, or metal ions or other modifications caused by changes in O₂ concentration or concentration of NO related species. The term “NO related species” is used herein to mean NOₓ where x is 1 or 2, NO⁻ and NO⁺ and organic derivatives thereof including nitrites and nitrates. The term “oxygen related species” is used herein to mean O₂ and reactive oxygen species, for example, superoxide, hydrogen peroxide or lipid peroxide.

The “oxidation state” of a protein is a measure of the level of oxidation of a protein or one or more amino acids in a protein. Examples of amino acid that may undergo oxidative modification include but are not limited to cysteine, methionine, arginine, and tryptophan. The level of oxidation may be determined by measuring the thiol pool in amino acids such as cysteine. Cysteine is more susceptible to oxidation-induced modification and provide a wide range of oxidized derivatives that reflect a range of ROS fluxes.

In some embodiments, the oxidation state comprises an oxidation state of a thiol pool. Thiols serve as redox buffers, redox signaling intermediates, and as oxidant stress markers. The sequence of redox reactions that govern oxidation of protein thiols begins with a monothiol protein (PrSH) that undergoes oxidation to the thiolate anion, and then to sulfenic, sulfinic acids and sulfonic acids. The first two steps are reversible oxidation steps and the last two are stable end-oxidation products, at least under physiologic conditions. In addition, protein monothiols can engage in thiol-disulfide exchange reactions with low molecular weight thiols (e.g., glutathione), which serves as an antioxidant thiol buffer. They can undergo modification through reaction with reactive nitrogen species, such as peroxynitrite, to form S-nitroso proteins, and they can undergo protein-protein disulfide exchange reactions to form mixed disulfides between proteins.

Vicinal dithiols are another important subgroup of the protein thiols, which are chemically reactive with each other, mainly due to their steric adjacency within the tertiary structure of the protein. Vicinal dithiols are important because these are the most sensitive indicators of oxidant stress within a protein. These are the first species to undergo oxidation, usually to vicinal disulfides, but occasionally to mixed disulfides.

The thiol pool may be measured by mass spectrometry. Mass spectrometry is an important tool in the identification of proteins and peptides. Using ESI or MALDI-MS, peptides can be ionized intact into the gas phase and their masses accurately measured. Based on this information, proteins can readily be identified using a methodology called protein mass mapping or peptide mass mapping, in which these measured masses are compared to predicted values derived from a protein database. Further sequence information can also be obtained by fragmenting individual peptides in tandem MS experiments. In addition, large scale changes in protein expression levels (protein profiling) between two different samples can be assessed using quantitative tools such as two-dimensional gel electrophoresis (2D-GE) or stable isotope labeling in conjunction with mass spectrometry measurement.

Sequence specific proteases or certain chemical cleaving agents are used to obtain a set of peptides from the target protein that are then mass analyzed. For example, the enzyme trypsin is a commonly used protease that cleaves peptides on the C-terminal side of the relatively abundant amino acids arginine (Arg) and lysine (Lys). Thus, trypsin cleavage results in a large number of reasonable sized fragments from 500 to 3000 Daltons, offering a significant probability for identifying the target protein. The observed masses of the proteolytic fragments are compared with theoretical “in silico” digests of all proteins listed in sequence database. The matches or “hits” are then statistically evaluated and marked according to the highest probability.

This strategy is predicated on the existence of the protein sequence within the database searched. The quality and content of such databases are continually improving as a result of genomic sequencing of entire organisms, and the likelihood for obtaining matches is now reasonably high. While exact matches are readily identified, proteins that exhibit significant homology to the sample are also often identified with lower statistical significance. This ability to identify proteins that share homology with poorly characterized sample species makes protein mass mapping a valuable tool in the study of protein structure and function.

Upon submitting a query to a search program, a theoretical digest of all the proteins in the database is performed according to the conditions entered by the researcher. Variables that can be controlled include taxonomic category, digestion conditions, the allowable number of missed cleavages, protein isoelectric point (pI) and mass ranges, possible post translational modifications (PTMs), and peptide mass measurement tolerance. A list of theoretical peptide masses is created for each protein in the database according to the defined constraints, and these values are then compared to the measured masses. Each measured peptide generates a set of candidate proteins that would produce a peptide with the same mass under the digestion conditions specified. The proteins in these sets are then ranked and scored based on how closely they match the entire set of experimental data.

This method of identification relies on the ability of mass spectrometry to measure the masses of the peptides with reasonable accuracy, with typical values ranging from roughly 5 to 50 ppm. The experimentally measured masses are then compared to all the theoretically predicted peptide digests from a database containing possibly hundreds of thousands of proteins to identify the best possible matches. Various databases are available on the Web, and can be used in conjunction with such computer search programs such as Profound (developed at Rockefeller University), ProteinProspector (University of California, San Francisco) and Mascot (Matrix Science, Limited). One obvious limitation of this methodology is that two peptides having different amino acid sequences can still have the same exact mass. In practice, matching 5-8 different tryptic peptides is usually sufficient to unambiguously identify a protein with an average molecular weight of 50 kDa, while a greater number of matches may be required to identify a protein of higher molecular weight. In some embodiments, mass measurements of the undigested protein could also be used for protein identification.

A more specific database searching method involves the use of partial sequence information derived from MS/MS data. Tandem mass spectrometry experiments allows peptide identification by yielding fragmentation patterns for individual peptide. Analogous to peptide mapping experiments, the experimentally obtained fragmentation patterns can be compared to theoretically generated MS/MS fragmentation patterns for the various proteolytic peptides arising from each protein contained in the searched database. Statistical evaluation of the results and scoring algorithms using search engines such a Sequest (ThermoFinnigan Corp) and MASCOT (Matrix Science, Limited) facilitate the identification of the best match. The partial sequence information contained in tandem MS experiments is more specific than simply using the mass of a peptide, since two peptides with identical amino acid contents but different sequences will exhibit different fragmentation patterns.

While the molecular weight information obtained from ESI, and MALDI are useful in the preliminary stages of characterization, it can also be important to gain more detailed structural information through fragmentation. Tandem mass spectrometry, the ability to induce fragmentation and perform successive mass spectrometry experiments on these ions, is generally used to obtain this structural information.

One of the processes by which fragmentation is initiated is known as collision-induced dissociation (CID). CID is accomplished by selecting an ion of interest with the mass analyzer and then subjecting that ion to collisions with neutral atoms or molecules. The selected ion will collide with the collision gas such as argon, resulting in fragment ions which are then mass analyzed. CID can be accomplished with a variety of instruments, most commonly using triple quadrupoles, quadrupole ion traps, Fourier transform-ion cyclotron resonance (FT-ICR) mass spectrometry (FTMS), time-of-flight reflectron and quadrupole time-of-flight mass analyzers. The triple quadrupole and quadrupole ion trap combined with electrospray are currently the most common means of generating peptide structural data, as they are capable of high sensitivity, and produce a reasonable amount of fragmentation information. MALDI with time-of-flight reflectron and Fourier transform-ion cyclotron resonance are also common sources for structural information.

In order to obtain peptide sequence information by mass spectrometry, fragments of an ion must be produced that reflect structural features of the original compound. Most peptides are linear molecules, which allow for relatively straightforward interpretation of the fragmentation data. The process is initiated by converting some of the kinetic energy from the peptide ion into vibrational energy. This is achieved by introducing the selected ion, usually an (M+H)+ or (M+nH)ⁿ+ion, into a collision cell where is collides with neutral Ar, Xe, or He atoms, resulting in fragmentation. The fragments are then monitored via mass analysis. Tandem mass spectrometry allows for a heterogeneous solution of peptides to be analyzed and then by filtering the ion of interest into the collision cell, structural information can be derived on each peptide from complex mixture.

Certain limitations for obtaining complete sequence information exist using tandem mass spectrometry. For example, in determining the amino acid sequence of a peptide, it is not possible for leucine and isoleucine to be distinguished because they have the same mass. The same difficulty will arise with lysine and glutamine since they have the same nominal mass, although high resolution tandem analyzers (quadrupole-TOF and FTMS) can distinguish between these amino acids.

When these methodologies cannot directly be used to identify all the proteins present in a typical biological sample due to the significant signal suppression caused by complex mixtures in mass spectrometry. Tryptic digestion of a typical protein can result in the production of roughly fifty peptides, while miscleavages and various PTMs can give rise to many other unique species. Thus, biologically-derived samples can contain thousands to literally millions of individual peptides in the case of whole cell extracts. By comparison, the tryptic digestion of approximately 3-5 proteins results in a peptide mixture complex enough to cause considerable signal suppression. Thus, in some preferred embodiments, samples of proteins (or peptides in a proteolytic digest) are separated by gel electrophoresis or liquid chromatography prior to mass analysis.

Gel electrophoresis is one of the most widely used techniques for separating intact proteins. In sodium dodecyl sulfate-polyacrylamide gel electrophoresis (SDS-PAGE), sometimes called one dimensional gel electrophoresis, the proteins are treated with the denaturing detergent SDS and loaded onto a gel. Upon application of an electric potential across the gel, the proteins migrate through the gel towards the anode at a rate inversely proportional to their size. Upon completion of the separation, the proteins may be visualized using any of a number of different staining agents (Coomassie, Sypro Ruby, or Silver), and the individual bands are physically excised from the gel. These excised spots are subjected to destaining, reductive alkylation, in-gel digestion, peptide extraction, and finally mass analysis for protein identification.

The combination of SDS-PAGE electrophoresis with an isoelectric focusing step also enables the separation of proteins of similar mass. In two-dimensional gel electrophoresis (2D-GE), proteins are first separated according to their isoelectric points (pI) by electrophoresis through a solution or gel containing an immobilized pH gradient, with each protein migrating to a position in the pH gradient corresponding to its isoelectric point. Once the isoelectric focusing step is complete, gel electrophoresis similar to SDS-PAGE is performed orthogonally to separate the proteins by size. Like 1D gels, 2D gel spots can be cut out, enzymatically digested, and mass analyzed for protein identification. Using this technique, thousands of proteins can simultaneously be separated and removed for identification.

In addition, 2D gels can help facilitate the analysis of certain PTMs. For example, differently phosphorylated forms of the same base protein may appear as a series of bands of roughly identical mass but different isoelectric points.

Automated liquid handling robots have been developed that perform all the sample preparation steps for peptide mapping experiments, including gel destaining, alkylation/reduction, in gel digestion, peptide extraction, and MALDI target plating.

Mass spectral data acquisition systems have similarly been automated to acquire spectra, process the raw data, and perform database searches for numerous samples. Commercial MALDI-TOF systems are available that can perform over 1,000 mapping experiments in just twelve hours. These systems are able to perform automated calibrations, vary laser energies, and adjust laser firing location to maximize signal, with the entire data acquisition process requiring approximately 30 seconds or less. Similarly, automated data processing systems can recognize suitable signals, identify monoisotopic peaks, and submit summary peak lists directly to a search engine.

Such high throughput proteomics systems enable the investigation of multiple unknown samples at once such as those coming from gels. Additionally, the flexibility of automated acquisition and data analysis software allows to rapidly reacquire and/or reanalyze entire batches of samples with minimal user effort. Automated systems are, however, limited in that they are only as good as the data provided. For example, the detection and accurate mass assignment of species exhibiting low signal-to-noise ratios is often poor. Such issues have led to the development of post-acquisition data processing. Improvements in these processes have enabled high through-put automated systems to achieve identification “hit” rates equal to or above those obtained normally.

An alternative approach to gel electrophoresis techniques involves the use of analytical separation methods such as high performance liquid chromatography (HPLC). Whereas gel electrophoresis techniques separate intact proteins, liquid chromatography can be performed on proteolytic peptides. One of the means of performing peptide LC-MS/MS involves the direct coupling of the LC to an ion trap mass spectrometer through an electrospray ionization interface. Other mass analyzers suitable for these experiments include triple quadrupoles and quadrupole time-of-flights.

The additional sequence information provided by tandem MS in the LC/MS experiments can be extremely powerful, sometimes enabling a definitive protein identification to be made on the basis of a single peptide. Generally, fragmentation information can be obtained for peptides with molecular masses up to 2500 Daltons. Larger peptides can reveal at least partial sequence information that often suffices to solve a particular problem.

LC-MS/MS methodologies for protein identification have been extended to mixtures of even greater complexity by performing multi-dimensional chromatographic separations before MS analysis. Extremely complex tryptic digests are first separated into a number of fractions using one mode of chromatography, and each of these fractions is then further separated using a different chromatographic method.

Protein profiling studies can also be performed using multi-dimensional LC-MS/MS in conjunction with stable isotope labeling methodologies. Two samples to be compared are individually labeled with different forms of a stable isotopic pair, and their tryptic digests are then combined before the final LC-MS analysis. This should result in every peptide existing as a pair of isotopically labeled species that are identical in all respects expect for their masses. Thus, each isotopically labeled peptide effectively serves as its partner's internal standard, and the ratio of the relative heights of two isotopically labeled species provides quantitative data as to any change that occurred in the protein from which the peptide arose.

Alternatively, methods of isotope-coded affinity tags (ICAT) provide an applicable approach based on the in vitro chemical labeling of protein samples. ICAT utilizes the high specificity of the reaction between the thiol groups and haloacetyls (such as iodoacetamide) to chemically label cysteine residues in proteins with isotopically light or heavy versions of a molecule that differ only by the existence of eight hydrogen or deuterium atoms, respectively. The labeled protein samples are then combined and simultaneously digested, resulting in every cysteine-containing peptide existing as an iostopically labeled pair differing in mass by eight Daltons per cysteine residue. The general strategy of chemical labeling can be extended to other functional groups present in proteins for which chemical selective reactions exist.

Examples of other methods to measure the thiol pool are listed in Table 1.

TABLE 1

| Oxidation State | Designation | Method |
|---|---|---|
| Thiolate | PrS | Biotinylated Iodoacetamide |
| | | (BIAM) |
| Vicinal Dithiol | Pr(SH)₂ | Biotinylated |
| | | Glutathionylacetyl)amino)phenyl- |
| | | arsine oxide (GSAO-B) |
| Sulfenic Acid | PrSOH | Dimedone |
| Glutathiolated | PrSSG | Anti-glutathione Ab |
| Disulfide | | |
| Glutathiolated | PrSSG | Biotinylated Glutathione |
| Disulfide | | monoethylester (Bio-GEE) |
| Interprotein | PrSSPr\acute{ } | Nonreducing f/b reducing two- |
| Disulfide | | dimensional gel PAGE |

The vicinal dithiol may be characterized using either sepahrose-aminohexaenoyl-4-aminophenylarsine oxide or, for greater sensitivity, biotinyl-4-(N—(S-glutathionyl-acetyl)amino) phenylarsine oxide (GSAO-B) (Prot Sci 9:2436-2445). After reacting the sample with this reagent, the Ps(SH)₂ can be isolated using a steptavidin affinity column.

Biotin-conjugated iodacetamide (BIAM) can be used to identify reactive cysteinyl residues as described by Kim et al. (Anal. Biochem 283:214-221, 2000). As cysteinyl residues are sensitive to oxidation by low concentrations of H₂O₂, they serve as potential early targets of low levels of ROS and may complement information obtained with vicinal dithiols. Sulfenic acid residues in protein (PrSOH) can be determined by treating the proteome with dimedone (Biochemistry 42:9906-9914; 2003) and the resulting derivatized proteins are determined by detecting a 141 mass unit shift in the mass spectra of the labeled peptide.

In some embodiments, determining the oxidation state of the protein involves measuring a level(s) of S-nitrosylation. This may be performed using a variation of the biotin switch method to detect S-nitrosoproteins in cells. 200 mM Methylmethanethiosulfonate (MMTS) is used to block thiols followed by 200 μM ascorbate to reduce PrSNO, after which the thiol derived from PrSNO is reacted with MMTS-biotin. An avidin affinity column may be used to isolate the PrSNo-containing proteins, and 2-D (two-dimensional) gel electrophoresis is performed. In-gel digests of protein spots may be performed with trypsin. Protein spots on the 2-D gel are excised and in-gel digestion is carried out with a Montage in-gel digestion kit (Millipore). Digests of protein bands are first analyzed by matrix-assisted laser desorption ionization-time-of-flight (MALDI-TOF) mass spectrometry and samples are subjected to micro-liquid chromatography-electron spray ionization-mass spectrometry/mass spectrometry (microLC-ESI-MS/MS) using a Q-TOF Ultima system (Waters, Milford, Mass.). MS/MS fragmentation spectra are analyzed using ProteinLynx software package.

According to one aspect of the invention, a method of diagnosing a disorder characterized or caused by oxidant stress in a subject is provided. The method involves determining an oxidation state in a protein and comparing the oxidation state in the protein to a control. In some embodiments, the protein is in a sample from the subject. The oxidation state of the protein may be determined by any of the methods described above. The invention also involves comparing the oxidation state in the protein to a control value. The control value can take a variety of forms. It can be single value (e.g., a cut-off value), such as a median or mean. It can be established based upon comparative groups. It can be a range, for example, where the tested population is divided equally (or unequally) into groups, such as a low-risk group, a medium-risk group and a high-risk group, or into quadrants. For example, the lowest quadrant oxidation state being individuals with the lowest risk of developing a disorder characterized/carried by oxidant stress and the highest quadrant being individuals with the highest risk of developing a disorder characterized/carried by oxidant stress. The control value can depend upon the particular population selected. The control value may take into account the category in which a subject(s) falls.

The following Example is provided to illustrate specific instances of the practice of the present invention and is not intended to limit the scope of the invention. As will be apparent to one of ordinary skill in the art, the present invention will find application in a variety of methods.

### EXAMPLE

The majority of protein disulfides in cells are considered an important inert structural, rather than a dynamic regulatory, determinant of protein function. We showed that some disulfides in proteins are also regulated by cell redox status with functional consequences. We found that ROS produced by mitochondria are actively utilized by cells to facilitate cell-surface protein disulfide formation, as well as folding and transport, in mammalian cells. Inhibition of mitochondrial ROS production suppressed protein disulfide formation and induced reductive stress leading to dysfunction and retention (possibly in the Golgi, in part) of a group of cell-surface disulfide-containing proteins. Sparsely cultured cells produced less ROS than confluent cells, which lead to decreased disulfide formation and decreased activity of a subgroup of disulfide-containing cell-surface receptors. These data support the concept of two subproteomes comprising the disulfide proteome, a structural group and a redox-sensitive regulatory group, the latter having direct functional consequences for the cell.

Disulfide bond formation is a critical event in protein synthesis and function. Recent studies showed that some protein disulfides form transiently in the cytosol (1, 2) as a reflection of cell redox state that affects protein function and cell phenotype. Disulfide exchange, catalyzed by the protein disulfide isomerase family, has been thoroughly studied; however, de novo formation of protein disulfide bonds in mammalian cells has been less well characterized (3). Only in recent years has the mechanism of protein disulfide formation emerged in other cell types in which oxidative enzyme catalysts are necessary for disulfide bond formation. Key effectors of disulfide formation include the ER resident thiol oxidase ERo1 (4, 5), an essential gene in yeast; and the disulfide regulatory system consisting of DsbB protein and the electron transport chain in E. coli (6). Homologs of Ero1 have been identified in mammalian cells, and their overexpression promotes intracellular disulfide formation (7-10). However, these proteins are not essential for mammalian cell survival, nor has it been demonstrated that they are the primary determinants of cellular disulfide formation. We found that reactive oxygen species produced by mitochondria are actively utilized by cells to facilitate cell-surface protein disulfide formation in mammalian cells. Our data support the concept of two subproteomes comprising the disulfide proteome, a structural group and a redox-sensitive regulatory group, the latter having direct functional consequences for the cell.

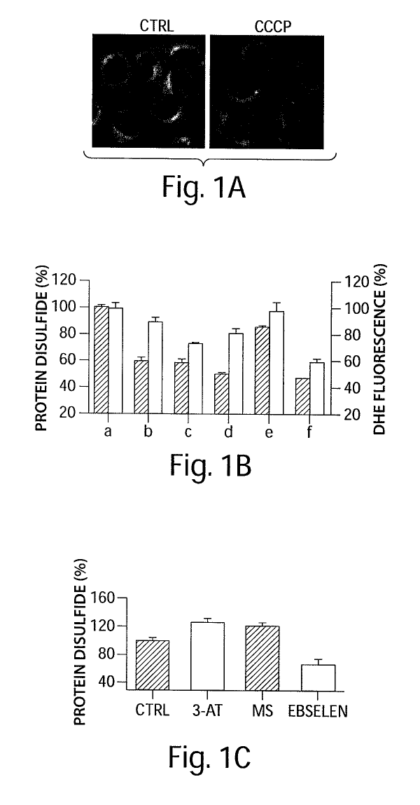

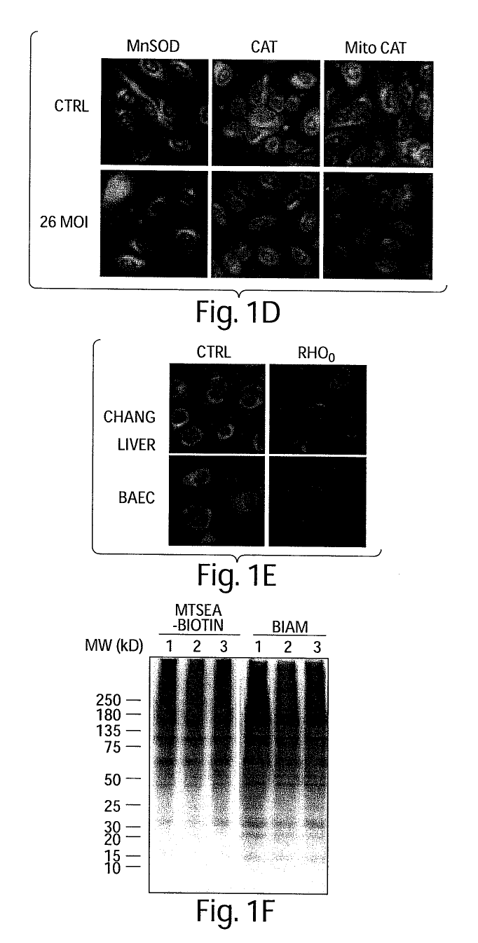

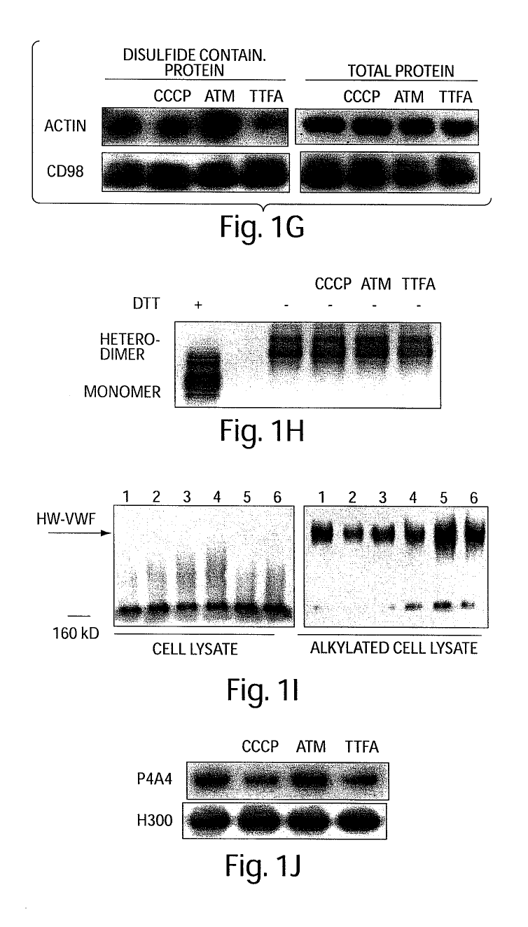

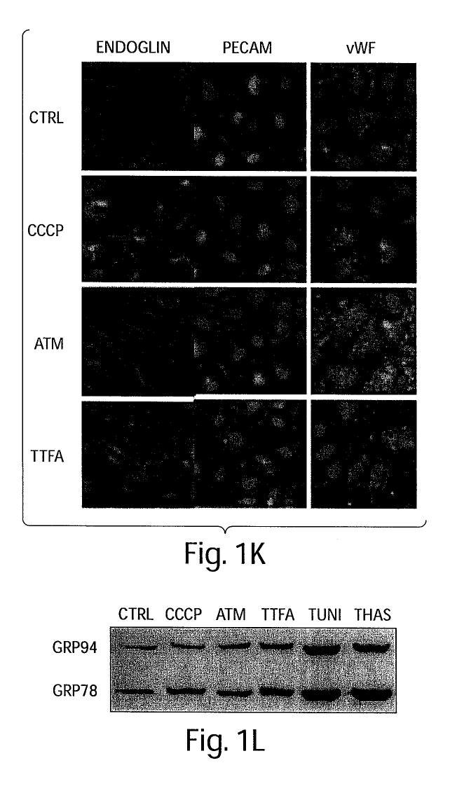

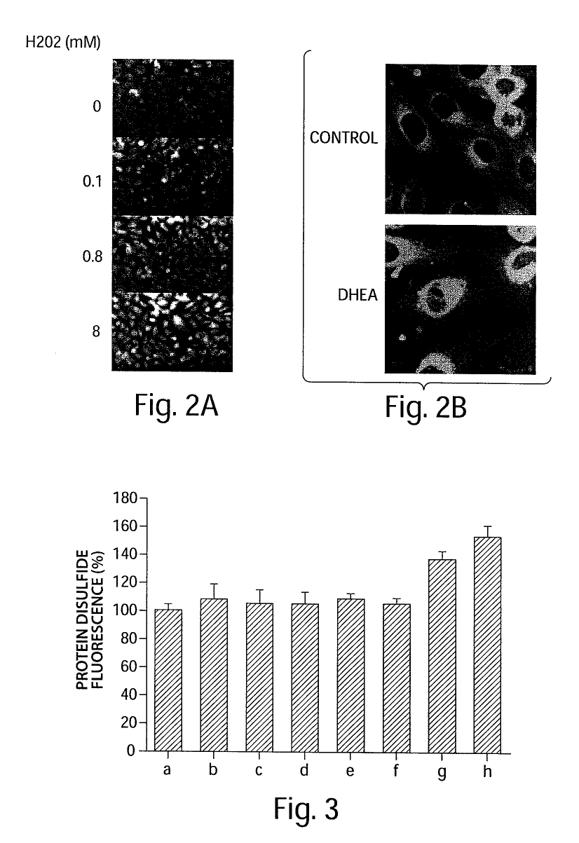

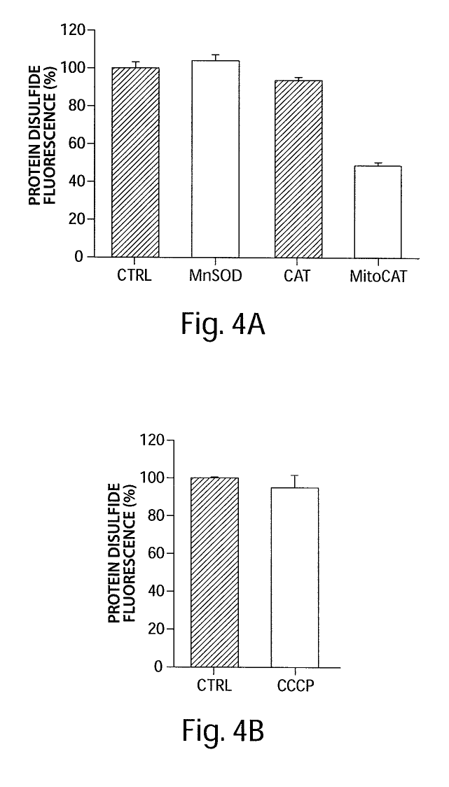

In order to quantify global protein disulfide status in cells, we established a specific method to image protein disulfides in situ, by first blocking protein thiols, then reducing the disulfides and fluorescently labeling the resulting thiols. Using this method in cultured cells, we observed a pattern consistent with localization to the Golgi apparatus (FIG. 1a,1k), while mitochondria also show a weak signal. The signal increased somewhat when cells were exposed to oxidative stress (FIG. 2). Affinity purification and identification of cellular disulfide-containing proteins by mass spectrometry (Table 2) reveal 9 proteins, 7 of which are cell-surface, membrane proteins known to contain disulfides, consistent with studies in plants (11) and E. coli (12) reporting that under normal growth conditions, most proteins containing disulfides are secreted or membrane-bound. Upon treatment with the mitochondrial uncoupler carbonylcyanide m-chlorophenylhydrazone (CCCP), the overall signal decreased (FIG. 1a), suggesting that the disulfide proteome depends on mitochondrial ROS generation. As shown in FIG. 1b, among all mitochondrial inhibitors tested, CCCP, rotenone, thienoyltrifluoroacetone (TTFA), and myxothiazol decreased protein disulfide formation, with CCCP being the most effective, and antimycin A (ATM), a mitochondrial inhibitor known to be incapable of blocking ROS generation, the least effective. The decrease in signal was accompanied by decreased superoxide generation, as measured by dihydroethidium (DHE) fluorescence. By contrast, inhibitors of other ROS generating enzymes did not alter cellular protein disulfide content (FIG. 3). Inhibition of the peroxidases, catalase, and glutathione peroxidase-1 (GPx-1) increased protein disulfide content by 20% (p<0.05) (FIG. 1c), while the GPx-mimetic ebselen inhibited disulfide formation by 55% (p<0.01). The role of mitochondria in protein disulfide formation is further supported by the fact that the disulfide formation in cells markedly decreased when catalase was overexpressed in mitochondria (MitoCAT), but not in peroxisomes (FIG. 1d, FIG. 4), and that pseudo-Rho₀ cells devoid of mitochondrial DNA demonstrated a much lower protein disulfide signal than cells with intact mitochondria (FIG. 1e). In MitoCAT overexpressing cells, CCCP no longer had any effect on protein disulfide formation (FIG. 4), indicating that CCCP treatment and MitoCAT overexpression decreased protein disulfide formation through the same mechanism, i.e., decreased mitochondrial ROS production.

Although there is a global decrease of protein disulfide content when mitochondrial ROS generation is inhibited (FIG. 1f), the disulfides in different proteins are differentially sensitive to cellular redox state changes induced by mitochondrial inhibition. Inhibition of mitochondrial ROS decreased the disulfide signal of actin, but not that of CD98 (FIG. 1g). We found that CCCP was associated with dissociation of disulfide-linked multimeric vWF (in endothelial cells) (FIG. 1n), while the heterodimer CD98 (in Chang liver cells) remained intact (FIG. 1g). The change of disulfide content in vWF was also confirmed by vWF immunofluorescence, which showed a decrease in signal in human pulmonary artery endothelial cells (HPAECs) in which mitochondrial respiration was inhibited, as the anti-vWF antibody used has much higher reactivity toward multimeric, disulfide-linked vWF than reduced vWF monomer (FIG. 1k). Interestingly, when HPAECs were treated with the glucose-6-phosphate dehydrogenase inhibitor dehydroepiandrosterone (DHEA) or the gamma-glutamylcysteine synthetase inhibitor buthionine sulfoximine (BSO) to induce oxidative stress, detectable multimeric vWF also increased (FIG. 1i).

With inhibition of mitochondrial ROS, membrane localization of endoglin (FIG. 1k), but not PECAM, was eliminated, while staining within the Golgi increased. The alteration of surface expression has also been reported for CD13 with a disulfide forming cysteine site-mutant (13). Similarly, previous studies showed that disulfide-bound proteins are reduced and reversibly retained in the ER in dithiothreitol (DTT)-treated cells, whereas proteins entering the secretory pathway are not (14). We observed that a monoclonal antibody to endoglin, P4A4, was sensitive to a disulfide-containing epitope. In a non-reducing immunoblot, less endoglin was detected by P4A4 antibody in CCCP- or TTFA-treated cells, while this difference disappeared when endoglin was detected using another rabbit polyclonal antibody H300 (FIG. 1j), suggesting that disulfide formation in endoglin depends on mitochondrial ROS. Such disulfide-dependent antibody recognition was also reported previously for the cell surface protein, CD13 (13).

Although CCCP markedly decreased protein disulfide content, it did not induce significant upregulation of GRP78 or GRP94 compared to tunicamycin and thapsigargin treatments (FIG. 11). The localization of disulfide staining to the Golgi, retention of disulfide-deficient proteins in the Golgi, and lack of upregulated ER stress markers in CCCP-treated cells suggest that these disulfide-deficient proteins are at least partially folded within the Golgi system, and raise the distinct possibility that some protein disulfides are formed in the Golgi rather than the ER (15) under normal conditions.

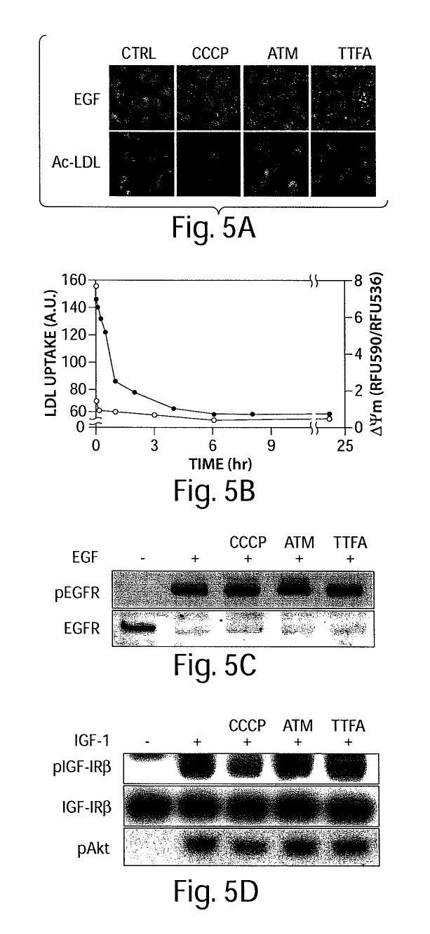

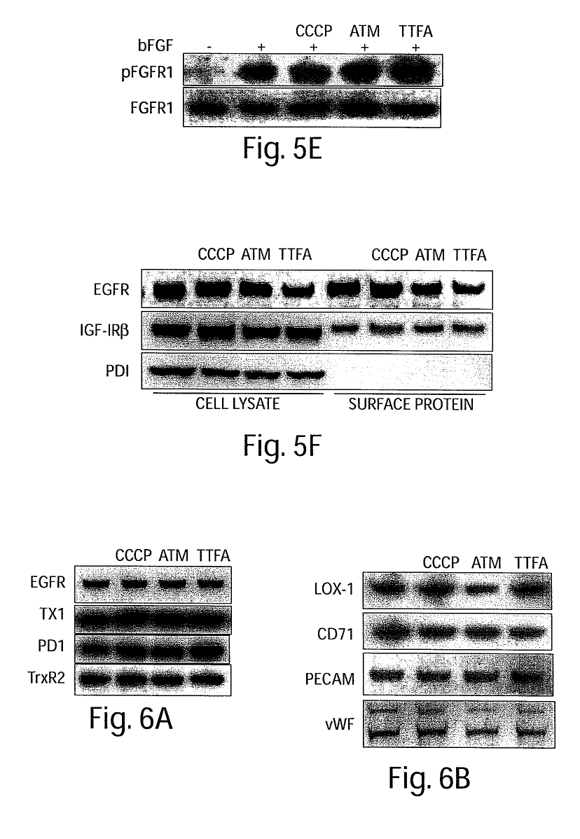

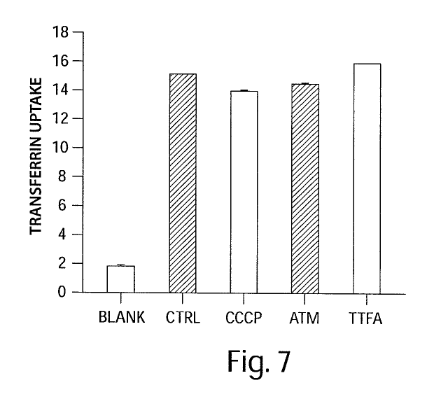

In order to assess the consequences of impaired disulfide bond formation on cell-surface receptor function, a group of cell surface disulfide-containing proteins was studied. The expression of all the receptors studied did not change with mitochondrial inhibitor treatment (FIG. 5, FIG. 6). Receptor-dependent uptake was not affected by mitochondrial inhibition for EGF (FIG. 5a) or transferrin (mediated by CD71) in HPAECs (FIG. 7). Interestingly, uptake of acetylated LDL (AcLDL) by HPAECs decreased after CCCP or TTFA, but not ATM, treatment. The loss of AcLDL uptake is a cumulative process, much slower than the immediate loss of mitochondrial membrane potential (MMP) after CCCP treatment (FIG. 5b), suggesting that it is not directly linked to MMP, but, rather, a kinetically slower downstream process, e.g., loss of a critical disulfide of the LOX-1 protein. With mitochondrial inhibition, folding and transport of these proteins was impaired; however, the correctly folded proteins already present on the cell surface can continue to function until their degradation and until new synthesis is exhausted over time.

In cells stimulated with the respective ligands, phosphorylation of IGF-1R and FGFR, but not EGFR, decreased after inhibition of mitochondrial ROS (FIG. 5c, d, e), despite the fact that there is no change in receptor expression levels. The loss of receptor function is explained by changes in localization of these receptors: there is much less expression of IGF-1R, but not EGFR (FIG. 5f), on the cell surface after mitochondrial inhibition, as yet another example of mitochondrial ROS redox-dependent translocation of disulfide-containing proteins. Receptors possessing intrinsic kinase domains were previously shown to be regulated by another ROS-mediated mechanism, in which the reversible inactivation of protein tyrosine phosphatases by ROS (16-21) is responsible for amplification of phosphorylation of the receptors. These mechanisms are excluded as explanations for our observations, as ligand-induced EGF phosphorylation was not dependent on mitochondrial respiration, consistent with a recent study (22).

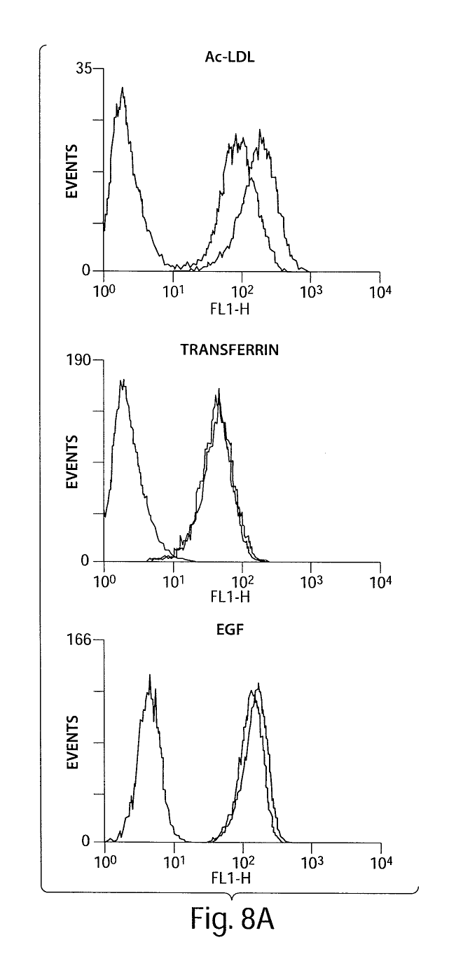

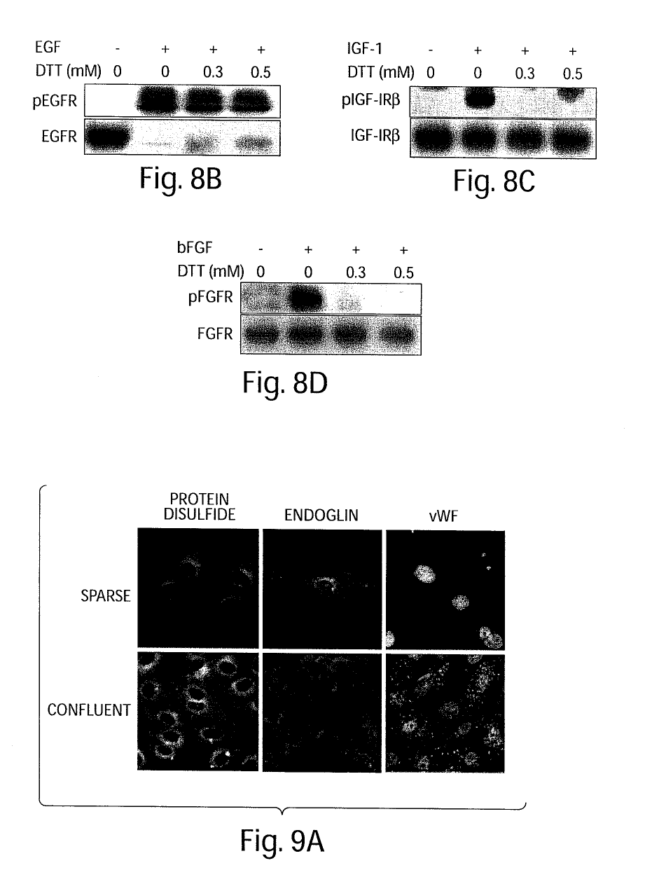

To confirm a direct disulfide-mediated effect of ROS on the function of these receptors, we also exposed cells to different concentrations of DTT to reduce disulfides directly. All receptors sensitive to mitochondrial inhibitors were also sensitive to DTT. As shown in FIGS. 8a and 8b, uptake of EGF or transferrin by cells did not change with 0.5 mM DTT treatment, while uptake of AcLDL by HPAECs decreased (FIG. 8a). Although there was a decrease of EGFR phosphorylation as detected by the phospho-specific antibody after DTT treatment, EGFR was completely undetectable (23) (EFGR immunodetection by Santa Cruz sc-03 antibody)(23), suggesting that EGFR is fully activated by EGF even in the presence of 0.5 mM DTT. By contrast, phosphorylation of IGF-1Rβ and FGFR were completely inhibited in the presence of 0.3 mM DTT (FIG. 8c, d).

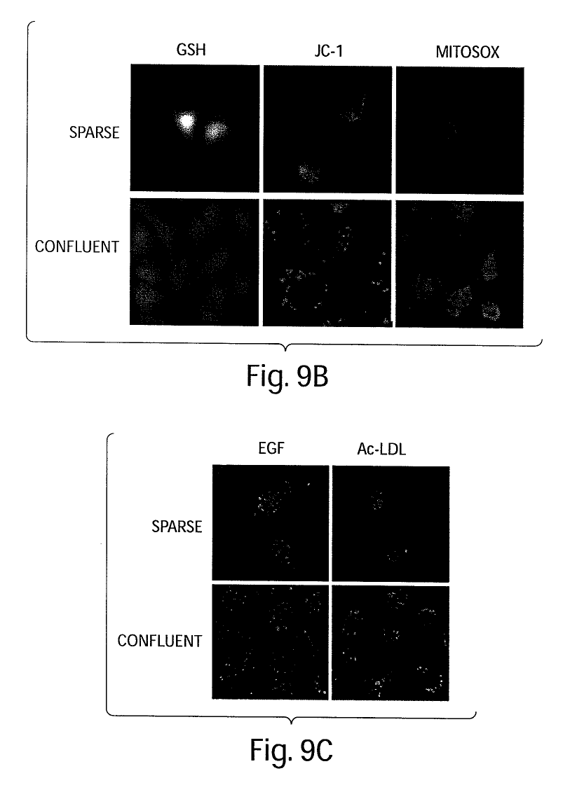

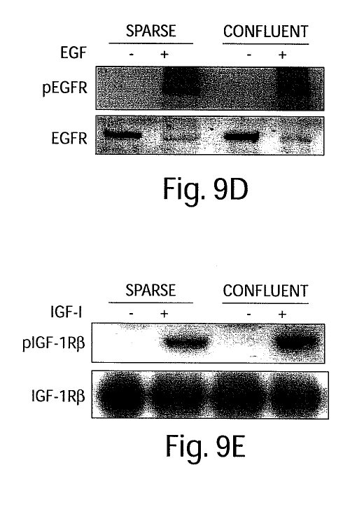

As shown in FIG. 9a, sparsely cultured cells show much less protein disulfide content than did confluent cells; in parallel with this observation, there is more intracellular GSH, lower MMP, and less mitochondrial superoxide generation in sparsely cultured than confluent cells (FIG. 9b), suggesting the existence of a more reductive state in sparsely cultured cells leading to less protein disulfide formation. Consequently, similar to observations in CCCP-treated cells, endoglin is localized to the Golgi rather than the cell surface in sparsely cultured cells. In addition, there is less multimeric vWF, (FIG. 9a), less uptake of AcLDL, and less ligand-induced phosphorylation of IGF-1Rβ in sparsely cultured cells, while EGF uptake EGF-induced phosphorylation of EGFR (FIG. 9c), and transferrin uptake were not affected by cell density. These data suggest that the alteration of receptor function in sparsely cultured cells may be a consequence of insufficient oxidative potential in these cells. The relationship of cell density to redox potential described here may also account, in part, for differences in bioactive nitric oxide in endothelial cells as a function of cell density (24). Supplementary Table 3 compares the sensitivity of the function of specific cell surface receptors to thiol reduction (DTT), mitochondrial electron transport inhibition (CCCP), and cell density.

In summary, we report here that mitochondria-derived ROS are actively utilized by cells to facilitate cell-surface protein disulfide formation, and, by implication, are important for protein folding and transport. Mammalian cells have different ways to handle de novo disulfide synthesis, with mitochondria as the main determinant. By contrast, yeast cells exclusively require Ero1p for disulfide formation (4, 5, 15, 25) and do not depend on mitochondrial respiration for disulfide formation (15). Use of hydrogen peroxide, usually a byproduct of mitochondrial respiration, for “structural disulfide” homeostasis in mammalian cells may provide an evolutionary advantage through improved energy efficiency.

The very strong link among disulfide formation, mitochondrial inhibition, reductive potential, and cell density indicates that even traditionally termed “structural disulfides,” the most abundant disulfides in the cell, are not equivalent with respect to their role in maintaining functional protein integrity. We, thus, define a subgroup of the disulfide proteome as “regulatory disulfides” to account for this dissociation between structural and functional integrity among disulfide-containing subproteome groups. Changes in cellular redox state have been shown to affect signaling globally by many groups; however, the mechanism by which this effect is mediated is largely unknown. Only a very few cell surface proteins, CD4 and integrins, have been shown to have cleavable disulfides that can act as functional switches (26). Here we report that cell redox regulation of the disulfide bond is much more pervasive than previously understood, providing a new perspective on how surface molecules are regulated by cell redox state consistent with the view that cells must maintain an appropriate redox balance to limit both oxidative and reductive stress for optimal protein function. This concept highlights a unique regulatory mechanism for and by a subgroup of the disulfide proteome in mammalian cells, and its potential consequences for protein function and cell phenotype.

### Material and Methods

Detection of cellular disulfide-containing proteins: We developed a method for imaging disulfide-containing proteins in situ. Methanol-fixed cells were treated with 200 mM iodoacetamide in 100 mM Tris, pH 8.3, 5 mM EDTA at 37° C. for 1 hour to block thiols. The cells were then washed six times with Tris-buffered saline, pH 8.0, 5 mM EDTA, after which they were incubated with 5 mM EDTA, 1 mM tris(2-carboxyethyl) phosphine, pH 8.3, at room temperature to reduce disulfides and with 1 mM 5-iodoacetamidofluorescein (5-IAF) in 100 mM Tris to label the resulting thiols for 1 hour. Excess dye was removed by washing the cells repeatedly with TBS. Stained cells fixed to slides were then treated with Prolonged Antifade Mounting Medium, and cell nuclei were counterstained with DAPI. Fluorescent images were taken with a Nikon fluorescence TE 2000 microscope. Fluorescence intensity was quantified by subtracting background fluorescence, then integrating the image with the NIH IMAGEJ program and normalizing by cell number as determined by DAPI fluorescence. Four fields magnified×20 were analyzed per experiment, with 100-200 cells counted per sample.

Proteomic identification of cellular disulfide-containing proteins: Disulfide-containing proteins in HPAECs were labeled by the method described above; however, 0.2 mM MTSEA-biotin-X or 1 mM biotinylated Iodoacetamide (BIAM) was used in place of 5-IAF. Biotin-labeled proteins were then isolated by avidin-D agarose gel affinity chromatography. Digests of proteins were subjected to microliquid chromatography electrospray ionization tandem MS (micro-LC-ESI-MS-MS) using a LCQ Deca XP system (Thermo Finnigan). MS-MS fragmentation spectra were analyzed using the Sequest software package.

Surface receptor functional assessment: After treatment, cells was incubated with 500 ng/ml Alexa 488-labeled EGF in DMEM containing 1% BSA, 50 μg/ml Alexa-488-labeled transferrin for 10 min, or 10 μg/ml Alexa 488-labeled AcLDL for 30 min in growth medium, and then washed with cold HBSS four times before fixation with formaldehyde at room temperature. Cells were also trypsinzed and ligand uptake quantified by flow cytometry (FACS Calibur). For protein phosphorylation studies, cell lysates were also collected 5 min after stimulation with ligands.

Reagents: Methyl methanethiosulfonate (MMTS) was purchased from Calbiochem, La Jolla, Calif. Glutathione, buthionine sulfoximine (BSO), L-arginine, ascorbate, Hepes, N-ethyl-maleimide, dichlorodihydrofluorescein diacetate (DCFDA), iodoacetamide (IAA), neocuproine, antimycin A (ATM), myxothiazol, sodium azide, thienoyltrifluoroacetone (TTFA), carbonylcyanide m-chlorophenylhydrazone (CCCP), L-nitroarginine methylester (L-NAME), nialamide, indomethacin, allopurinol, xanthine oxidase, 1-aminobenzotriazole, apocynin, thapsigargin, tunicamycin, tris(2-carboxyethyl) phosphine (TCEP), rotenone, uridine, ethidium bromide, dithiothreitol (DTT), dehydroepiandrosterone (DHEA), and dimethylsulfoxide (DMSO) were obtained from Sigma, St. Louis, Mo. 2-((6-((biotinoyl)amino)-hexanoyl) amino)ethylmethanethiosulfonate (MTSEA biotin-X) and biotinylated iodoacetamide (BIAM) were purchased from Biotium (Hayward, Calif.). Avidin-D agarose gel was obtained from Vector Laboratories, Burlingame, Calif. Dihydroethidine bromide (DHE), MitoSox (JC-1), Prolonged Antifade kit, DAPI, Hoechst 33342, 5-iodoacetamidofluorescein (5-IAF), fluorescein-5-maleimide-labeled wheat germ agglutinin (WGA), fluorescein-5-maleimide-labeled Con A (concanavalin A), Alexa 488-labeled acetylated LDL, Alexa 488-labeled EGF, Texas Red-labeled transferrin, and epidermal growth factor were obtained from Molecular Probes, Solon, Ohio. Bis-Tris Gel and MOPS-SDS running buffer, Silverquest silver stain kit, DMEM, penicillin, streptomycin, and fetal bovine serum (FBS) were purchased from Invitrogen, Carlsbad, Calif. Biorad DC protein assay and Biosafe Coomassie blue stain were obtained from Bio-Rad, Hercules, Calif. Basic fibroblast growth factor (bFGF) and insulin-like growth factor (IGF-1) were purchase from R & D, Minneapolis, Minn.

Cell culture: Bovine aortic endothelial cells (BAECs), human pulmonary aortic endothelial cells (HPAECs), and EGM-2MV media were obtained from Cambrex (San Diego, Calif.). Chang liver cells were obtained from ATCC. BAECs and Chang liver cells were grown in DMEM supplemented with 10% FBS, penicillin (100 units/ml), and streptomycin (100 μg/ml). HPAECs were grown in full EGM-2MV medium. To generate cells devoid of functional mitochondria, so-called pseudoRho0 cells, BAECs or Chang liver cells were incubated in DMEM containing ethidium bromide (100 ng/ml) and uridine (100 ug/ml) for 10 days and two passages before experimentation. To overexpress antioxidant enzymes, cells were incubated with 25 MOI adenovirus containing MnSOD, CAT, or MitoCAT overnight, then further cultured for 36 hrs before protein disulfide staining.

Reactive oxygen species detection: Cells were washed with PBS twice and incubated with medium containing DHE for 1 hr. Extracellular dye was removed by washing with PBS three times. Fluorescence was measured on a SpectraMax Gemini XPS fluorescence plate reader. To measure mitochondria-generated superoxide, cells were incubated with Mitosox in medium for 10 min, then washed and incubated in medium for 1 hr before fluorescence microscopy.

Cell surface thiol and intracellular GSH staining: Cells were washed with ice-cold DPBS and incubated with DMEM with 10 uM fluorescein-5-maleimide for 10 min on ice. Unbound dye was removed by washing with cold DPBS. Cells were then fixed with formaldehyde before microscopy. For Intracellular GSH staining, cells were incubated with 20 uM monobromobimane in HBSS for 10 min at 37° C. Unbound dye was removed by washing with HBSS. For negative controls, cells were incubated with 100 uM N-ethyl-maleimide for 10 min to alkylate GSH before labeling.

Organelle staining: Fixed cells were incubated with DPBS for 5 min, then with 1% BSA in DPBS for 10 min. Cells were briefly washed with DPBS, then incubated with fluorescently labeled lectins (WGA or Con A) to label subcellular organelles (Golgi apparatus or endoplasmic reticulum, respectively) for 50 min, after which they were, again, washed four times with DPBS.

Mitochondrial membrane potential measurement: Cells were incubated with medium containing 1 ug/ml JC-1 for 30° C., washed with HBSS, and then observed by fluorescence microscopy immediately. Alternatively, fluorescence was measured with a GEMINI XPS microplate reader, with an excitation wavelength of 488 nm and an emission wavelength of 536 nm, as well as an excitation wavelength of 528 nm and an emission wavelength 590 nm. Mitochondrial membrane potential was expressed as the ratio of emission at 590 nm to that at 536 nm.

Western blotting and indirect immunofluorescence: Cells were lysed in cell lysis buffer (Cell Signaling, Danvers, Mass.). Lysate protein concentration was measured by the Biorad DC protein assay. Equal amounts of proteins were separated by MOPS-SDS-PAGE, then transferred to poly(vinylidene difluoride) membranes (Invitrogen, Carlsbad, Calif.). Membranes were then blocked and the signal detected with a BM Chemiluminescence Blotting kit (Roche Diagnostics, Basel, Switzerland), BM POD precipitating blotting substrate (Roche Diagnostics, Basel, Switzerland), or Supersignal West Femto blotting kit (Pierce, Rockford, Ill.) according to the manufacturer's instructions. For protein inter-subunit disulfide bond detection, cells were lysed in cell lysis buffer containing 200 mM N-ethyl-maleimide and incubated at 37° C. for 1 hr. LDS sample buffer with or without DTT was added to cell lysates, and proteins were separated by MOPS-SDS-PAGE. For indirect immunofluorescence studies, formaldehyde-fixed cells were permeablized with 0.3% Triton for 10 min, blocked with 1% BSA for 30 min, then detected with antibodies. Primary antibodies used in this study include monoclonal or polyclonal antibodies against CD98, PECAM, endoglin, vWF, PDI, pEGFR, EGFR, FGFR, and CD71 from Santa Cruz; against actin, from Sigma, St. Louis, Mo.; against pFGFR, pIGF1R, and IGF1R from Cell Signaling (Danvers, Mass.); against thioredoxin, MnSOD, catalase, and GAPDH from Abcam, Cambridge, Mass.; and against LOX-1 from Serotec, Raleigh, N.C. All secondary fluorescent-labeled or horseradish peroxidase-conjugated antibodies were obtained from Jackson Immunoresearch, West Grove, Pa.

TABLE 2
Disulfide-containing proteins identified
in HPAECs by mass spectrometry

| Protein | Peptides identified |
|---|---|
| PECAM | 4 |
| Endoglin | 4 |
| CD9 | 2 |
| Actin | 2 |
| MCAM(melanoma cell adhesion molecule) | 2 |
| CD13 (aminopeptidase N) | 1 |
| CD98 | 1 |
| Na+/K+ ATPase beta 3 subunit | 1 |
| Integrin 5alpha. | 1 |

TABLE 3
Relation of disulfide formation to cell-surface receptor function

| | Sensitivity to | Sensitivity | Sensitivity to |
|---|---|---|---|
| | DTT | to CCCP | cell density |
| EGFR | − | − | − |
| FGFR | + | + | ND. |
| IGF-1R | + | + | + |
| CD71 | − | − | − |
| LOX-1 | + | + | + |
| vWF | + | + | + |
| Endoglin | N.D. | + | + |

### REFERENCES

1. Jakob U, Muse W, Eser M, & Bardwell J C (1999) Cell 96, 341-352.
2. Lee C, Lee S M, Mukhopadhyay P, Kim S J, Lee S C, Ahn W S, Yu M H, Storz G, & Ryu S E (2004) Nat Struct Mol Biol 11, 1179-1185.
3. Bardwell J C (2004) Nat Struct Mol Biol 11, 582-583.
4. Frand A R & Kaiser C A (1998) Mol Cell 1, 161-170.
5. Pollard M G, Travers K J, & Weissman J S (1998) Mol Cell 1, 171-182.
6. Bader M, Muse W, Ballou D P, Gassner C, & Bardwell J C (1999) Cell 98, 217-227.
7. Mezghrani A, Fassio A, Benham A, Simmen T, Braakman I, & Sitia R (2001) Embo J 20, 6288-6296.
8. May D, Itin A, Gal O, Kalinski H, Feinstein E, & Keshet E (2005) Oncogene 24, 1011-1020.
9. Gess B, Hofbauer K H, Wenger R H, Lohaus C, Meyer H E, & Kurtz A (2003) Eur J Biochem 270, 2228-2235.
10. Pagani M, Fabbri M, Benedetti C, Fassio A, Pilati S, Bulleid N J, Cabibbo A, & Sitia R (2000) J Biol Chem 275, 23685-23692.
11. Lee K, Lee J, Kim Y, Bae D, Kang K Y, Yoon S C, & Lim D (2004) Electrophoresis 25, 532-541.
12. Leichert L I & Jakob U (2004) PLoS Biol 2, e333.
13. Firla B, Arndt M, Frank K, Thiel U, Ansorge S, Tager M, & Lendeckel U (2002) Free Radic Biol Med 32, 584-595.
14. Lodish H F & Kong N (1993) J Biol Chem 268, 20598-20605.
15. Tu B P, Ho-Schleyer S C, Travers K J, & Weissman J S (2000) Science 290, 1571-1574.
16. Bae Y S, Sung J Y, Kim O S, Kim Y J, Hur K C, Kazlauskas A, & Rhee S G (2000) J Biol Chem 275, 10527-10531.
17. Bae Y S, Kang S W, Seo M S, Baines I C, Tekle E, Chock P B, & Rhee S G (1997) J Biol Chem 272, 217-221.
18. Meng T C, Fukada T, & Tonks N K (2002) Mol Cell 9, 387-399.
19. Lee S R, Yang K S, Kwon J, Lee C, Jeong W, & Rhee S G (2002) J Biol Chem 277, 20336-20342.
20. Rhee S G, Bae Y S, Lee S R, & Kwon J (2000) Sci STKE 2000, PE1.
21. Choi M H, Lee I K, Kim G W, Kim B U, Han Y H, Yu D Y, Park H S, Kim K Y, Lee J S, Choi C, et al. (2005) Nature 435, 347-353.
22. Chen K, Albano A, Ho A, & Keaney J F, Jr. (2003) J Biol Chem 278, 39527-39533.
23. EGFR immunodetection by Santa Cruz sc-03 antibody was totally eliminated after EGF stimulation, suggesting not only that all EGF receptors localized on the cell surface respond to EGF, but also that phosphorylation of the receptor and/or occupancy with ligand inhibits anti-EGFR antibody binding.
24. Zollner S, Aberle S, Harvey S E, Polokoff M A, & Rubyani G M (2000) Endothelium 7, 169-184.
25. Frand A R & Kaiser C A (2000) Mol Biol Cell 11, 2833-2843.
26. Hogg P J (2003) Trends Biochem Sci 28, 210-214.
27. Yang Y & Loscalzo J (2005) Proc Natl Acad Sci USA 102, 117-122.

All of the references and/or publications referred to herein are incorporated herein by reference in their entirety. In the event of conflicting disclosures the present detailed description is controlling.

We claim:

1. A method of identifying an oxidation-modified protein wherein an amino acid has undergone an oxidation-induced modification comprising:
(a) determining an oxidation state of the protein;
(b) exposing the protein to a condition that results in oxidation-induced modification of an amino acid in the protein;
(c) determining the oxidation state of the protein after exposure to the condition in (b); and
comparing the oxidation state of the protein before exposure to the condition in (b) to the oxidation state after exposure to the condition in (b), wherein if the oxidation state of the protein determined in (c) is greater than the oxidation state of the protein determined in (a), the protein is identified as an oxidation-modified protein.

2. The method of claim 1 further comprising identifying an oxidatively-modified peptide in the oxidation-modified protein by mass spectrometry.

3. The method of claim 1, wherein the oxidation state comprises an oxidation state of a thiol pool.

4. The method of claim 3, wherein the thiol pool comprises a protein vicinal dithiol (PR(SH)₂) pool, a protein glutathiolated (PrSSG) pool, or an interprotein disulfide pool (PrSSPr′) in the sample.

5. The method of claim 1, wherein the oxidation state comprises a level of S-nitrosylation.

6. The method of claim 1, wherein the oxidation state comprises a level of cysteine oxidation.

7. The method of claim 1, wherein the amino acid is cysteine, methionine, arginine, or tryptophan.

8. The method of claim 1, wherein the oxidation-induced modification comprises a modification generated in a disease/disorder (or is associated with a disease/disorder).

9. The method of claim 1, wherein the condition that results in an oxidation-induced modification comprises hydrogen peroxide, superoxide, peroxynitrite or perchlorate.

10. A method of diagnosing a disorder or predicting the risk of developing a disorder characterized/caused by oxidant stress in a subject, comprising:
determining an oxidation state in a protein,
comparing the oxidation state in the protein to a control, wherein an increase in the oxidation state compared to the control indicates that the subject has or is at risk of developing a disorder characterized/caused by oxidant stress.

11. The method of claim 10, wherein the disorder is an inflammatory disorder, an auto-immune disorder, a cardiovascular disorder, or insulin-independent diabetes mellitus (type II diabetes).

12. The method of claim 11, wherein the inflammatory disorder is allergic rhinitis, ankylosing spondilitis, arthritis, asthma, Behcet syndrome, bursitis, chronic obstructive pulmonary disease (COPD), Churg-Strauss syndrome, dermatitis, gout, Henoch-Schonlein purpura, inflammatory bowel disease (Crohn's disease or ulcerative colitis), inflammatory neuropathy, Kawasaki disease, myositis, neuritis, pericardits, polyarteritis nodosa, polymyalgia rheumatica, prostatitis, psoriasis, radiation injury, sarcoidosis, shock, sytemic inflammatory response syndrome (SIRS), Takayasu's arteritis, temporal arteritis, thromboangiitis obliterans (Buerger's disease), vasculitis, or Wegener's granulomatosus.

13. The method of claim 11, wherein the autoimmune disorder is Addison's disease, chronic thyroiditis, dermatomyositis, Grave's disease, Hashimoto's thyroiditis, hypersensitivity pneumonitis, insulin-dependent diabetes mellitus (type I diabetes), multiple sclerosis, myasthenia gravis, organ transplantation, pernicious anemia, Reiter's syndrome, rheumatoid arthritis, Sjogren's syndrome, systemic lupus erythematosis (SLE), thyroiditis, or urticaria.

14. The method of claim 11, wherein the cardiovascular condition is coronary artery disease, ischemic cardiomyopathy, myocardial ischemia, ischemic or post-myocardial ischemia revascularization, diabetic retinopathy, diabetic nephropathy, renal fibrosis, hypertension, atherosclerosis, arteriosclerosis, atherosclerotic plaque, atherosclerotic plaque rupture, cerebrovascular accident (stroke), transient ischemic attack (TIA), peripheral artery disease, arterial occlusive disease, vascular aneurysm, ischemia, ischemic ulcer, heart valve stenosis, heart valve regurgitation or intermittent claudication.

15. The method of claim 10, wherein the protein is in a sample from the subject.

16. The method of claim 15, wherein the sample is sample is blood, serum, plasma, urine, sputum, saliva, stool, cerebrospinal fluid, peritoneal fluid, cell, tissue, or a secretion.

17. A method of screening for an agent that modulates the oxidation state of a protein, comprising:
(a) determining an oxidation state of the protein;
(b) exposing the protein to an agent or a condition that results in an oxidation-induced modification of an amino acid side in the protein:
(c) determining the oxidation state of the protein after exposing the protein to the agent; and
comparing the oxidation state of the protein in (b) to the oxidation state of the protein in (a), wherein if the oxidation state of the protein determined in (b) is greater than the oxidation state of the protein determined in (a), the agent or condition is an oxidant or a pro-oxidant, and if the oxidation state of the protein determined in (b) is less than the oxidation state of the protein determined in (a), the agent or condition is an anti-oxidant.
